(19) United States
(12) Patent Application Publication
(10) Pub. No.: US 2023/0380441 A1
(43) Pub. Date: Nov. 30, 2023

(54) CONFECTIONERY PRODUCT

(71) Applicant: Perfetti Van Melle S.P.A., Lainate (Milano) (IT)

(72) Inventors: Güven Marangoz, KT Sittard (NL); Joaquin Antonio Lizano Iglesias, KT Sittard (NL)

(73) Assignee: Perfetti Van Melle S.P.A., Lainate (Milano) (IT)

(21) Appl. No.: 18/249,000

(22) PCT Filed: Oct. 20, 2021
(86) PCT No.: PCT/IB2021/059650

§ 371 Date: Apr. 13, 2023

(30) Foreign Application Priority Data
Oct. 20, 2020 (IT) 102020000024775

(51) Int. Cl. A23G 3/54; A23L 27/30; A23G 3/42; A23P 10/28; A61K 9/20; A61K 35/741

(52) U.S. Cl. CPC A23G 3/54; A23L 27/34; A23G 3/42; A23P 10/28; A61K 9/2054; A61K 35/741

(57) ABSTRACT

Disclosed is a process for the manufacture of a confectionary product consisting of a tablet characterised by the use of water-soluble ingredients.

The invention relates to a tablet comprising water-soluble ingredients, without giving rise to the formation of lumps which could prevent or interfere with the compression process.

The tablet according to the invention presents an outer surface which is visually varied due to the combination of granules of different colours.

### PRIOR ART

Some known tablets are made by compressing granules, particularly granules consisting of sweeteners, as indicated, for example, in US2002016518, which discloses the use of very small granules and does not require the use of hydrocolloids in the binder used for granulation, but the use of a sorbitol syrup.

EP329977 also discloses the preparation of tablets by compressing large granules, which undergo a granulation process using syrups as binders.

Both EP329977 and US2002016518 involve the use of irregularly-shaped sweetener granules.

EP452262 discloses tablets containing powdered active ingredients such as colorants, flavourings and sweeteners, which are added to pre-formed polydextrose granules.

Said prior art documents involve making the tablet by mixing, before compression, granular ingredients which have been separately pre-formed.

The techniques described above present some drawbacks, because they involve drying steps designed to remove the water content so as to prevent the formation of clumps of granules that would prevent a correct flow of material to the compression chamber.

The techniques described above also require the granules of the various ingredients of the tablet to be pre-mixed.

A further technical problem inherent in said process is that it is difficult to verify the formulation of the mixture introduced into the individual moulds, because the distribution of the various ingredients, being random, is not homogeneous, with the consequence that the tablets may contain different amounts of active ingredients.

Moreover, said techniques do not allow the manufacture of granules which are perfectly spherical and have the desired dimensions; on the contrary, the granules have irregular shapes and sizes.

Said irregularities mean that gaps are generated between one granule and another at the time of compression, with the undesirable effect of generating an excessively friable tablet. Moreover, said irregularities have an adverse effect on the flow of material, which does not glide optimally towards and into the compression chamber.

Another drawback associated with the techniques mentioned so far, which require little or no water, is that it is impossible to use water-soluble active ingredients.

A granulation technique characterised by use of a larger starting granule, coated with smaller granules using an aqueous binder, is known.

Said technique, used in the manufacture of medicaments, enables multilayer, spherical granules of the desired size to be made. However, even with the use of said technique, the problem of using water-soluble active ingredients in the manufacture of confectionery products has so far remained unsolved.

### DESCRIPTION OF THE INVENTION

It has now been discovered that the problems of the prior art can be overcome by compressing substantially spherical granules consisting of a core of sweetener having a suitable particle size, coated with particles of the same or another sweetener having a smaller particle size than the core, said particles being caused to adhere to the core by an aqueous solution of a suitable hydrocolloid and of at least one water-soluble active ingredient.

“Particle size” here refers to the average size of the particles or granules constituting an aggregate, determined by sieving.

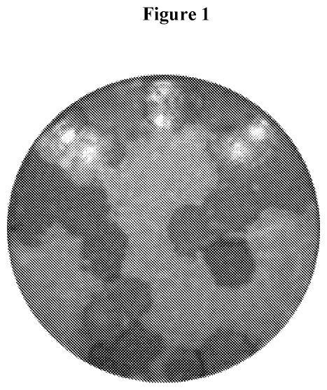

It has also been found that if granules of different colours are used, tablets with a visually variegated outer surface can be obtained. The resulting surface has a very pleasing, entirely novel visual effect, which can be described as a mosaic effect (FIG. 1).

Surprisingly, tablets obtained from spherical granules of different colours present a smooth surface with no gaps.

Demarcated polygonal shapes, perfectly interlocking with one another, are visually identifiable, each shape being characterised by the colour of the starting granules.

The mosaic effect also allows a further step of verification of the manufacture and final composition of the product, as the colours of the granules can be correlated with the specific active ingredient of the granule. For example, red granules could contain vitamins, while yellow granules could contain mineral salts. This means that the presence of the ingredients included in the product formula, and stated or displayed on the product packaging, can be verified, not only during manufacture but also by the end user.

The ingredients belonging to the various granules constituting the tablets according to the invention are therefore not mixed with one another but segregated, without gaps, into adjacent regions which originate from the starting granules. This makes it possible to include otherwise incompatible ingredients in the same tablet.

The invention therefore provides a tablet obtained by compressing substantially spherical granules of a plurality of colours consisting of a sweetener core having a particle size ranging between 400 and 600 μm, preferably about 500 μm, coated with particles of the same or another sweetener which have a particle size smaller than that of the core, preferably about 100 μm, said particles being caused to adhere to the cores by an aqueous solution of a hydrocolloid, optionally containing a water-soluble active ingredient.

Preferably, the sweetener is sorbitol and the hydrocolloid is hydroxypropyl methylcellulose.

Other polyols, such as isomalt, mannitol and xylitol, can be used.

The tablets according to the invention contain suitable conventional fillers, particularly fillers suitable for direct compression such as sugar, dextrose, powdered milk, spray-dried lactose, microcrystalline cellulose, dicalcium phosphate and other known additives.

The granules used for compression typically have a diameter ranging from 1.8 mm to 2.2 mm, preferably 2 mm.

The water-soluble active ingredient can be any compound used in the dietary, pharmaceutical or nutraceutical fields. Examples of classes of active ingredients comprise colorants, flavourings, vitamins, minerals, syrups, medicaments, plant extracts, amino acids, probiotics, prebiotics, inactivated bacteria and high-intensity sweeteners.

The sweetener constitutes 90 to 99% by weight of the total tablet weight, the hydrocolloid 0.1% to 0.5% by weight thereof and the other ingredients 1 to 10% by weight thereof.

The weight ratio of the core granules to the smaller sweetener particles typically ranges from 1/3 to 1/5.

The different colours of the granules can be determined by adding colouring agents permitted for use in foodstuffs or by the natural colour of the active ingredients or fillers included in the composition of the granules.

Tablets obtained by compressing granules of two, three or four colours are preferred.

The tablets and confectionery products made from the tablets can be prepared by a process comprising:
a) treatment of sweetener cores having a particle size ranging from 400 to 600 μm and of particles of one or more sweeteners, preferably sorbitol, having a smaller particle size, with an aqueous solution of a hydrocolloid and a water-soluble active ingredient to give substantially spherical granules with a diameter ranging from 1.8 mm to 2.2 mm, preferably 2 mm;
b) one or more repetitions of step a) with cores and particles of different colours from that of the granules resulting from step a), and with aqueous solutions of hydrocolloid containing a different water-soluble active ingredient;
c) mixing of the granules obtained in a) and b) and addition of a lubricant to the mixture;
d) distribution of the mixture between moulds, and compression.

To optimise the compression step, the granules selected for the manufacture of the tablet are mixed with one another together with a lubricant, preferably magnesium stearate.

The resulting tablet will typically consist of 99.75% granules and 0.25% lubricant. The following examples illustrate the preparation of single-layer tablets obtained by compressing granules having three different colours.

The same invention can be used to make one or more layers of multilayer tablets.

### EXAMPLE 1

Single-Layer Tablet with Granules of 3 Different Colours.

TABLE 1

| No. | Ingredient | Percentage (%) without water |
|---|---|---|
| 1 | Sorbitol | 33% |
| 2 | Mint flavouring | 0.16% |
| 3 | Aspartame | 0.04% |
| 4 | Colour 1 | 0.002% |
| 5 | HPMC | 0.06% |
| 6 | Sorbitol | 33% |
| 7 | Mint flavouring | 0.16% |
| 8 | Aspartame | 0.04% |
| 9 | Colour 2 | 0.002% |
| 10 | HPMC | 0.06% |
| 11 | Sorbitol | 33% |
| 12 | Mint flavouring | 0.16% |
| 13 | Aspartame | 0.04% |
| 14 | Colour 3 | 0.002% |
| 15 | HPMC | 0.06% |
| 16 | Magnesium stearate | 0.25% |
| | Total without water | 100% |

The tablet of Example 1 is obtained by using three different sets of granules. The granules belonging to the first set, characterised by colour 1, have a particle size of 2 mm, and are obtained from ingredients 1 to 5; the granules belonging to the second set, characterised by colour 2, have a particle size of 2 mm, and are obtained from ingredients 6 to 10; the granules belonging to the third set, characterised by colour 3, have a particle size of 2 mm, and are obtained from ingredients 11 to 15.

The granules belonging to the three sets are spherical, and are mixed in a tumbler, to which magnesium stearate is added.

The mixture of the three sets of granules and magnesium stearate is then conveyed to the moulds of a rotary tablet press, wherein it is compressed to obtain tablets containing the ingredients listed in Table 1 with their percentages.

The tablets have a single layer characterised by a smooth surface wherein demarcated areas of a polygonal shape, each characterised by one of the colours indicated as colour 1, colour 2 and colour 3, are distinguishable.

### EXAMPLES 2-4

Examples 2-4 describe tablets made according to the composition of Table 2; however, each example is made with granules of a different particle size.

TABLE 2

| | Example no. | | |
|---|---|---|---|
| | Example 2 | Example 3 | Example 4 |
| Granule | 1.8 mm | 2 mm | 2.2 mm |
| particle size | | | |
| Evaluation | The tablets present a | The tablets present a | The tablets present a |
| | pattern of regularly | pattern of regularly | pattern of nearly always |
| | alternating white, green | alternating white, green | regularly alternating |
| | and red areas, indicating | and red areas, indicating | white, green and red |
| | the balanced presence of | the balanced presence of | areas, indicating the |
| | vitamin C, iron bisglycinate | vitamin C, iron bisglycinate | balanced presence of |
| | and pyridoxine hydrochloride. | and pyridoxine hydrochloride. | vitamin C, iron bisglycinate |
| | The areas have polygonal | The areas have well-defined | and pyridoxine hydrochloride. |
| | outlines which are nearly | polygonal outlines and are | The areas have well-defined |
| | always well-defined, and | clearly distinct from each | polygonal outlines and are |
| | are clearly distinct from | other. | clearly distinct from each |
| | each other. | | other. |

TABLE 3

| No. | Ingredient | Percentage (%) without water |
|---|---|---|
| 1 | Sorbitol | 32.500% |
| 2 | Mint flavouring | 0.160% |
| 3 | Aspartame | 0.040% |
| 4 | Sodium ascorbate | 0.500% |
| 5 | HPMC | 0.060% |
| 6 | Sorbitol | 32.800% |
| 7 | Mint flavouring | 0.160% |
| 8 | Aspartame | 0.040% |
| 9 | Iron bisglycinate | 0.200% |
| 10 | HPMC | 0.060% |
| 11 | Sorbitol | 33.000% |
| 12 | Mint flavouring | 0.150% |
| 13 | Aspartame | 0.040% |
| 14 | Allura red | 0.002% |
| 15 | Pyridoxine hydrochloride | 0.010% |
| 16 | HPMC | 0.060% |
| 17 | Magnesium stearate | 0.218% |
| | Total without water | 100% |

The tablets of Examples 2-4 are obtained by using three different sets of granules, each characterised by a different water-soluble active compound and a different colour. The granules belonging to the first set, characterised by the presence of sodium ascorbate, are naturally white and obtained from ingredients 1 to 5 (Table 3); the granules belonging to the second set, characterised by the presence of iron bisglycinate, are naturally green and obtained from ingredients 6 to 10 (Table 3); the granules belonging to the third set, characterised by the presence of pyridoxine hydrochloride (vitamin B6), are coloured red with allura red colouring, and obtained from ingredients 11 to 16 (Table 3).

The tablets of Examples 2-4 have the same composition; a daily portion of 6×700 mg tablets provides 23% of the NRV (Nutrient Reference Value) of vitamin C, 16% of the NRV of iron, and 25% of the NRV of vitamin B6.

The tablets of examples 2-4 are differentiated from one another by the size of the granules from which they are obtained. All the granules are obtained from sorbitol cores, granulated with sorbitol of a smaller size.

The tablets are obtained from granules belonging to the three sets described in Example 1. The size of all three sets in each example is the same, as reported in Table 2. The resulting tablets were qualitatively evaluated by a group of four experts on the basis of their appearance, according to whether the presence of the various functional ingredients was observed.

1. A tablet obtained by compressing substantially spherical granules of a plurality of colours consisting of a sweetener core having a particle size ranging from 400 to 600 μm, coated with particles of the same or another sweetener having a particle size smaller than that of the core, said particles being caused to adhere to the cores by an aqueous solution of a hydrocolloid optionally containing a water-soluble active ingredient.

2. A tablet according to claim 1 wherein the sweetener is sorbitol and the sweetener core has a particle size of 500 μm.

3. A tablet according to claim 1 wherein the hydrocolloid is hydroxypropyl methylcellulose.

4. A tablet according to claim 1, wherein the granules have a diameter ranging from 1.8 mm to 2.2 mm.

5. A tablet according to claim 1, wherein the water-soluble active ingredient is selected from colorants, flavourings, vitamins, minerals, syrups, medicaments, plant extracts, amino acids, probiotics, prebiotics, inactivated bacteria and high-intensity sweeteners.

6. A confectionery product comprising a tablet according to claim 1.

7. The tablet according to claim 1, wherein the sweetener core is coated with particles having a particle size about 100 μm.

8. A tablet according to claim 1, wherein the granules have a diameter of 2 mm.
